(19) United States
(12) Patent Application Publication
(10) Pub. No.: US 2009/0099146 A1
(43) Pub. Date: Apr. 16, 2009

(54) Compositions Containing Sterol-derived Esters

(75) Inventors: Peter Horlacher, Bellenberg (DE); Dieter Hietsch, Illertissen (DE); Wolfgang Albiez, Illertissen (DE); Franz Timmermann, Illertissen (DE); Katja Beck, Bellenberg (DE)

Correspondence Address: FOX ROTHSCHILD LLP, 2000 MARKET STREET, PHILADELPHIA, PA, 19103, US

(73) Assignee: Cognis IP Management GmbH, Duesseldorf (DE)

(21) Appl. No.: 12/282,172

(22) PCT Filed: Feb. 27, 2007
(86) PCT No.: PCT/EP2007/001656

§ 371 Date: Sep. 8, 2008

(30) Foreign Application Priority Data
Mar. 8, 2006 (DE) 10 2006 011 090.0
Sep. 1, 2006 (DE) 10 2006 041 302.4

(51) Int. Cl. A61K 31/56; A61P 3/06

(52) U.S. Cl. 514/170

(57) ABSTRACT

The invention relates to sterol fatty acid esters of average chained fatty acids, having a chain length of 8 and 10 carbon atoms in a fatty acid distribution C₈ to C₁₀ (weight ratio) from 95:5 to 5:95, preferably, fatty acid distributions of C₈ to C₁₀ (weight ratio) from 90:10 to 20:80 and in particular, distributions of the fatty acids C₈ to C₁₀ (weight ratio) from 64:36 to 50:50 or from 78:22 to 65:35, are used. Preferably, capric and/or caprylic acids are used as fatty acids. Said sterol compounds can be easily incorporated into food, in particular, into drinks and milk products based on their wettability and easy to melt properties at low temperatures without using complex equipment, and have good organolepetic and sensory properties, and improved stability in relation to commercial sterol esters having long-chained and unsaturated fatty acids.

### CROSS REFERENCE TO RELATED APPLICATIONS

This application is the National Phase entry of PCT/EP2007/001656, filed Feb. 27, 2007, which claims priority to German patent application numbers DE 102006011090.0, filed Mar. 8, 2006, and DE 102006041302.4, filed Sep. 1, 2006, each of which are incorporated herein by reference in their entireties.

### FIELD OF THE INVENTION

This invention relates generally to sterol derivatives and compositions containing sterol derivatives, and to the use of such compounds and compositions in foods, cosmetics pharmaceutical preparations, and the like and to the use of such compounds, compositions and preparations for the production of cholesterol-lowering agents.

### BACKGROUND OF THE INVENTION

The use of cholesterol-lowering sterols, stanols and sterol/stanol derivatives in food preparations has acquired increasing significance in recent years. The literature offers numerous formulation options for enabling poorly soluble phytosterols and phytostanols to be incorporated in food preparations, cosmetic or pharmaceutical products. Besides leading to poor dispersibility, the unfavorable solubility behavior of the substances reduces their bioavailability and adversely affects the stability of the food preparations. Published literature describes how the availability of sterols can be improved by reducing the particle sizes, mainly by micronization. However, the reduction of particle size and the resulting surface enlargement in turn give rise to poor processability because the energy-rich particles aggregate and show very poor wettability.

Accordingly, it has heretofore been generally necessary to use emulsifiers to distinctly improve the dispersion properties. Even though food emulsifiers are distinguished by good compatibility and have already been known for some time, efforts are being made to reduce the quantity of emulsifiers or even to avoid them altogether because emulsifiers can also influence the bioavailability of other substances present in the foods or can adversely affect the stability of the formulations.

In addition, the incorporation of emulsifiers still frequently requires further technically imaginative formulation developments to minimize the disadvantages of poor further processing. Although sterol/emulsifier complexes enable the sterols to be easily and directly incorporated in food preparations, the reduced sterol content of the formulations has a negative effect because the increase in the quantities used also increases the input of emulsifiers.

An alternative to the pure sterols or stanols is to use derivatives esterified with fatty acids in foods, as shown by way of example in European patents EP 0 898 896 B1, EP 0 911 385 B1 and EP 1 075 191 B1, because sterol esters are comparable with the sterols in their cholesterol-lowering effect, and in pharmaceutical preparations (EP 0 435 682 B1). Sterols and stanols with fatty acids having chain lengths of 2 to 26 carbon atoms are disclosed. Commercially available sterol and stanol esters are generally derivatives with fatty acids from vegetable oils, such as for example sunflower oil, rapeseed oil, linseed oil, rice bran oil, safflower oil or soybean oil and, accordingly, mainly contain relatively long-chain saturated and unsaturated fatty acids with chain lengths of 16 to 22 carbon atoms. The esterified derivatives are a little easier to incorporate by virtue of their higher solubility. As long ago as 1907, M. F. M. Jaeger described some short-chain sterol esters in regard to their physico-chemical properties (M. F. M. Jaeger, Recueil der Travaux Chimiques des Pays-Bas et de la Belgique, 26 (1907), pp. 311 to 356). The choice of the fatty acids in such case may have an important influence on the properties of the various sterol derivatives in regard to melting point, stability and solubility, as already shown in U.S. patent U.S. Pat. No. 3,751,569 and in European patent EP 1 075 191 B1. The disadvantage of esters of relatively long-chain fatty acids is their poor processability which is largely attributable to their high melting point. Although sterol esters of unsaturated fatty acids are easier to process, foods to which these sterol derivatives have been added show poor stability. In addition, the percentage content of total sterol in the formulation decreases with increasing chain length of the fatty acids, so that larger quantities of the esters have to be incorporated to achieve a high sterol content in the food. By contrast, low-melting esters with short-chain fatty acids have the disadvantage of poor organoleptic and sensory properties which make the foods containing them unpleasant to consume.

Accordingly, one problem addressed by certain aspects of the present invention was the problem of providing a sterol-based composition that was capable of at once having favorable sensory and organoleptic properties, the ability to be readily incorporated into foods, and high stability as formed and in foods.

### SUMMARY OF THE INVENTION

One aspect of the present invention provides certain sterol-derived compounds and compositions comprising certain sterol derivatives, such as sterol esters and stanol esters. In certain preferred embodiments, the sterol-derived compounds of the present invention are esters of sterols and/or esters of sterol derivates, and even more preferably in certain embodiments medium chain fatty acid esters of sterol and/or sterol-derivates, such as medium chain fatty acid esters of stanol. The present invention also includes compositions containing such compounds and the uses and methods of using such compounds in a wide variety of applications, including in connection with food, beverages, dietary preparations, dietary supplements, cosmetics, medicaments, pharmaceutical preparations, and the use of one or more of these compounds and/or compositions for the production of cholesterol lowering agents.

### DETAILED DESCRIPTION OF THE EMBODIMENTS

In certain preferred embodiments, the present invention provides sterol-derived fatty acid esters, preferably of medium-chain fatty acids, and even more preferably with a chain length of 8 and/or 10 carbon atoms. In certain highly preferred embodiments, the present composition comprise at least two sterol-derived fatty-acid esters in which at least a first compound comprises a fatty acid moiety having 8 carbon atoms and at least a second compound comprises a fatty acid moiety having 10 carbon atoms, preferably with a distribution of C₈ to C₁₀ (ratio by weight) of from about 95:5 to about 5:95. Fatty acid distributions of C₈ to C₁₀ (ratio by weight) of from about 90:10 to about 20:80 are also preferably used in many embodiments, with distributions of the C₈ to C₁₀ fatty acids (ratio by weight) of from about 64:36 to about 50:50, or from about 78:22 to about 65:35 being particularly preferred in certain embodiments.

As used herein, the term “sterol-derived” refers to and includes within its meaning sterol compounds and compounds that are formed from or otherwise structurally related to sterols. Thus, for example, the term “sterol-derived” compound includes within its scope not only all sterols (including those that have not been modified) and stanols, which are formed by the hydrogenation of sterols.

Taking impurities and other fatty acids into account, the ratio by weight of about 64:36 to about 50:50 encompasses the distribution of C₈ to C₁₀ fatty acid moieties in preferred embodiments of about 60:40 while the ratio of about 78:22 to about 65:35 encompasses in certain preferred embodiments the distribution of C₈ to C₁₀ of about 70:30. Unbranched and branched, saturated and unsaturated fatty acids, preferably including those selected from the group consisting of n-octanoic acid, n-decanoic acid, ethylhexanoic acid and common nations of two or more of these are suitable and preferred in certain embodiments. The saturated unbranched fatty acids caprylic acid (n-octanoic acid) and/or capric acid (n-decanoic acid) are particularly preferred in certain embodiments.

It will be appreciated that from the standpoint of commercial production, fatty acids may frequently consist of mixtures of different chain lengths so that the sterol-derived esters produced from them in accordance with the present invention may frequently contain small quantities of fatty acids with other chain lengths than the preferred C₈ and C₁₀. The sterol-derived fatty acid esters according to certain preferred embodiments of the invention contain not greater than about 10% by weight, preferably not greater than about 5% by weight and, even more preferably, not greater than about 3% by weight other fatty acids than C₈ and C₁₀, based on the weight of the total fatty acids present in the ester.

The preferred sterol-derived fatty acid esters of the present invention, and particularly sterol fatty acid esters and the stanol fatty acid esters have a very low melting point which enables them to be melted at low temperatures and directly incorporated in the food. Even the use of pure sterol octanoic acid ester and, in particular, pure sterol decanoic acid ester in foods has considerable advantages when it comes to processing in foods. If mixtures of both fatty acids are selected for the esterification of the sterols, esters with even lower melting points are obtained. Even with fatty acid distributions of C₈ to C₁₀ of from about 90:10 to about 20:80, melting points of not greater than about 70° C. are obtained. Even temperature-sensitive and water-containing food preparations, which may be heated only briefly and only to relatively low temperatures, may be used as a basis for many of the preferred sterol-derived fatty acid esters of the present invention. These sterol derivatives are also particularly suitable for processing in beverages and milk products. Fatty acid distributions of C₈ to C₁₀ of from about 64:36 to about 50:50, or from about 78:22 to about 65:35 have highly advantageous processing properties because the desirably low melting point depression of the mixtures as against the pure fatty acids has been observed in the case of such distributions, particularly for sterol fatty acid asters. The fatty acid mixture preferably contains not greater than about 10% by weight, more preferably not greater than about 5% by weight and, even more preferably not greater than about 3% by weight fatty acids with other chain lengths.

The foods to be produced need only be heated to at most 60 to 75° C. during processing and the molten sterol-derived ester in accordance with the present invention can be very thoroughly distributed simply by stirring. During the subsequent cooling phase, it is present in the food in very fine and homogeneous distribution. Apart from a slight taste of the medium-chain sterol-derived fatty acid, the food thus produced has very good organoleptic and sensory properties by virtue also of the extremely fine distribution.

Besides a neutral taste and favorable sensory properties, the high total sterol content achieved in the foods thus enriched through the high percentage sterol content of the ester is particularly advantageous for the choice of medium-chain fatty acids. Compared with conventional sterol esters with long-chain fatty acids, the sterol derivatives according to the invention have a far higher content of pure sterol and enable the total quantity of sterol derivative used to be reduced.

The sterol-derived esters according to the invention contain at least about 60% by weight, preferably at least about 70% by weight and, in a particularly preferred embodiments, at least about 75% by weight total sterol or sterol-derived moiety, based on the weight of the sterol-derived compound.

In caprylic acid (n-octanoic acid) and capric acid (n-decanoic acid), fatty acids present in the form of the medium-chain triglycerides (MCTs)—also known as Miglykol®—esterified with glycerol are preferably used for the esterification. Since MCT oils in the field of human nutrition reduce the uptake of fats and increase both the burning of fats and the metabolism rate, sterol-derived esters of these fatty acids could also produce other main and secondary effects of value in terms of nutrition physiology.

The sterol-derived esters according to preferred embodiments of the invention have a solid consistency which contributes towards good processing because such a property provides for easy dosing, portioning and packaging. In addition, the esters with medium-chain, saturated fatty acids provide for good storage because they are considerably more stable to oxidation than conventional sterol esters based on sunflower oil, rapeseed oil, linseed oil, rice bran oil, safflower oil or soybean oil which mainly contain relatively long-chain saturated and unsaturated fatty acids having chain lengths of 16 to 22 carbon atoms. In preferred embodiments of the invention, the sensory properties of the compounds and/or the compositions of the present invention are not impaired by the usual storage and/or transportation conditions. The sterol derivative according to the preferred embodiments of the invention can be stored under standard conditions (RT) and can even be transported at elevated temperatures, as encountered in Asiatic countries and/or in summer (30-40° C.), without any damage to its properties. This high oxidation stability is of course also reflected in improved stability of the correspondingly enriched food product.

Accordingly, sterol-derived fatty acid esters of medium-chain fatty acids with a chain length of 8 and/or 10 carbon atoms in a fatty acid distribution of C₈ to C₁₀ of 100:0 to 0:1000 are eminently suitable for the production of cholesterol-lowering agents.

Sterols obtained from plants and vegetable raw materials—so-called phytosterols and phytostanols belonging to the groups of 4-desmethyl sterols, 4-monomethyl sterols and 4,4-dimethyl sterols—are preferably used in accordance with the present invention. Known examples of 4-desmethyl sterols include ergosterol, brassica sterol, campesterol, avenasterol, desmosterol, clionasterol, stigmasterol, poriferasterol, chalinosterol, sitosterol and mixtures thereof. Of these, β-sitosterol, stigmasterol, campesterol and brassica sterol are preferably used. Vegetable raw material sources for the sterols include inter alia seeds and oils of soybeans, canola, palm kernels, corn, coconut, rape, sugar cane, sunflower, olive, cotton, soya, peanut or products from the production of tall oil. Sterols from the production of tall oil and rape sterols are preferably used.

Mixtures of different sterols and stanols are obtained, depending on the raw material source. The main component of the sterols is many preferred embodiments is β-sitosterol. The sterol mixtures used in the production of the present compounds and compositions may also contain relatively large quantities of campesterol and stigmasterol and the hydrogenated derivatives sitostanol and campestanol, and may also contain quantities, preferably small quantities, of brassica sterol and preferably less than about 3% of other sterols and stanols.

The sterol-derived compounds of the present invention also include in certain embodiments the hydrogenated forms of the sterols, so-called stanols. Thus, besides the sterol fatty acid esters of medium-chain fatty acids, the corresponding esters of the hydrogenated derivatives, the stanol fatty acid esters of medium-chain fatty acids, preferably with a chain-length of 8 and 10 carbon atoms in a fatty acid distribution of C₈ to C₁₀ (ratio by weight) of from about 95:5 to about 5:95, with a fatty acid distribution of C₈ to C₁₀ (ratio by weight) of from about 90:10 to about 20:80 being preferred in certain embodiments, and distributions of the C₈ to C₁₀ fatty acids (ratio by weight) of from about 64:36 to about 50:50 or from about 78:22 to about 65:35 being particularly preferred in certain embodiments, particularly in connection with processing in food products.

Production can be carried out by standard methods, for example by esterification of sterol-derived compounds, including for example mixtures of various sterols and/or stanols with saturated medium-chain C₈ and C₁₀ fatty acids. Esters of the fatty acids can also be esterified by transesterification. Esterification with the corresponding anhydrides or acid halides is also possible. Corresponding processes can be found in the literature.

The preferred sterol-derived fatty acid ester compounds and compositions according to the invention eliminate the need for emulsifiers when it comes to incorporation in foods in many embodiments. For example, it is contemplated that the present compounds and compositions may readily be incorporated in foods selected from the group consisting of spreading fats, margarine, butter, vegetable oils, frying fats, peanut butter, mayonnaise, dressings, cereals, bread and confectionery products, cakes, wheat bread, rye bread, toast, crispbread, ice cream, puddings, milk products, yogurt, cottage cheese, cream, candies, chocolate, chewing gum, muesli bars, milk beverages, soya beverages, fruit juices, vegetable juices, fermented beverages, noodles, rice, sauces, cheese, spreading cheese, meat and sausages.

Since, in preferred embodiments, the foods to be produced only have to be gently heated during processing and since the preferred molten sterol-derived ester is very uniformly distributed simply by stirring and is present in very fine and homogeneous distribution during the subsequent cooling phase, water-containing and temperature-sensitive food products, such as beverages and milk products, for example milk, milk beverages, whey and yogurt beverages, fruit juices, fruit juice mixtures, fruit juice beverages, vegetable beverages, still and sparkling beverages, soya milk beverages and protein-rich liquid food substitute beverages and fermented milk preparations, yogurt, drinking yogurt, or cheese preparations, are particularly suitable as a basis for the sterol-derived esters according to the invention.

Accordingly, the present invention relates to food products containing the sterol-derived fatty acid esters according to the invention. The sterol-derived compounds are preferably used in beverages and milk products, which then contain from about 0.1 to about 50% by weight, preferably from about 0.5 to about 20% by weight and even more preferably from about 0.5 to about 3% by weight of sterol-derived esters, based on the total weight of the foods, and in fat-based products in which preferably from about 1 to about 25% by weight, and more preferably from about 5 to about 10% by weight, based on the total weight of the foods, are incorporated.

### EXAMPLES

### Example 1

### Production of the Sterol Ester with Medium-Chain Fatty Acids C8/C10 (70/30)

726 g of a fatty acid containing 8 to 10 carbon atoms (Edenor® C8-70, Cognis: 0.5% by weight C₆, 69-75% by weight C₈, 23-27% by weight C₁₀, 2% by weight C₁₂) were introduced into a reaction vessel and heated under nitrogen to 120° C. 1120 g tall oil sterol (Arboris® Sterols AS-2™, Arboris) and 480 g rape sterol (Generol 98 RF, Cognis) were then slowly added in three portions, the temperature being kept above 100° C. The reactor contents were then heated for 3 hours to 210° C., the upper phase of the reaction distillate being continuously returned to the reaction mixture. The mixture was then evacuated to 100 mbar and stirred for 4 hours. The excess fatty acid was then distilled off at 15 mbar and the reaction mixture was cooled to 90° C. and purged with nitrogen. The mixture was dried for 30 minutes at 85° C./<30 mbar before purging with nitrogen. The concluding purification step was carried out at 190° C./3 mbar by introduction of stripping steam (0.2 g per minute). 1911 g of an odorless, light, sensorially neutral, high-melting solid and 16 g of a yellow clear distillate were obtained as residue.

The sterol ester thus produced with medium-chain fatty acids having a chain length distribution of ca. 70% by weight C₈ and ca. 30% by weight C₁₀ has improved stability in relation to commercially available fatty acid esters with sunflower oil or rapeseed oil fatty acids (predominantly linoleic acid, oleic acid and small quantities of palmitic acid and stearic acid) and improved organoleptic properties and improved processability in relation to pure sterol.

### Gas Chromatographic Analysis of the Sterol Ester Produced in Example 1:

Analysis (GC analysis) of the sterol ester produced in Example 1 with medium-chain fatty acids having a chain length distribution of ca. 70% by weight C₈ and ca. 30% by weight C₁₀ revealed the following composition:

| | | |
|---|---|---|
| Total free sterol | 4.8 | area-% |
| Total free fatty acid | <<0.1 | area-% |
| Total sterol | 78.4 | wt.-% |
| Sterol ester | 94.9 | area-% |

Oxidation stability was evaluated by the Rancimat method:

| | |
|---|---|
| Temperature: | 120° C. |
| Air: | 20 l/h |
| Sample quantity: | 5.0 g |

Result:

| | |
|---|---|
| Sterol ester of Example 1 induction period | IP 39.3 h |
| Comparison (sterol ester based on sunflower oil fatty acid) | IP 6.5 h |

### Example 2

### Production of the Sterol Ester with Medium-Chain Fatty Acids C8/C10 (60/40)

782 g of a fatty acid containing 8 to 10 carbon atoms (Edenor® V 85, Cognis: max 1% by weight C₆, 54-64% by weight C₈, 36-45% by weight C₁₀, max. 2% by weight C₁₂) were introduced into a reaction vessel and heated under nitrogen to 120° C. 1120 g tall oil sterol (Arboris® Sterols AS-2™, Arboris) and 480 g rape sterol (Generol 98 RF, Cognis) were then slowly added in three portions, the temperature being kept above 100° C. The reactor contents were then heated for 3 hours to 210° C., the upper phase of the reaction distillate being continuously returned to the reaction mixture. The mixture was then evacuated to 100 mbar and stirred for 4 hours. The excess fatty acid was then distilled off at 15 mbar and the reaction mixture was cooled to 90° C. and purged with nitrogen. The mixture was dried for 30 minutes at 85° C./<30 mbar before purging with nitrogen. The concluding purification step was carried out at 190° C./3 mbar by introduction of stripping steam (0.2 g per minute). 1968 g of an odorless, light, sensorially neutral, high-melting solid (Mp.: 57.7° C.) were obtained as residue.

The sterol ester thus produced with medium-chain fatty acids having a chain length distribution of ca. 68% by weight C₈ and ca. 40% by weight C₁₀ also has improved stability in relation to commercially available fatty acid esters with sunflower oil or rapeseed oil fatty acids (predominantly linoleic acid, oleic acid and small quantities of palmitic acid and stearic acid) and improved organoleptic properties and improved processability in relation to pure sterol.

### Gas-Chromatographic Analysis of the Sterol Ester Produced in Example 2:

GC analysis revealed the following composition:

| | | |
|---|---|---|
| Total sterol | 76.5 | wt.-% |
| Cholesterol | 0.1 | area-% |
| Brassica sterol | 1.0 | area-% |
| Campesterol | 8.6 | area-% |
| Campestanol | 0.7 | area-% |
| Stigmasterol | 0.6 | area-% |
| β-Sitosterol | 78.3 | area-% |
| β-Sitostanol | 8.9 | area-% |
| Other sterols, total | 1.9 | area-% |
| Total free sterol | 1.4 | wt.-% |
| Sterol ester | ca. 97.0 | area-% |
| Total free fatty acid | <0.1 | area-% |

### Example 3

### Organoleptic Properties

An organoleptic test of the products was carried out by dissolving (50% solution) the sterol esters produced and comparison products in oil (MCT oil, Delios®, Cognis) at 40° C. and subjecting the solutions to organoleptic testing. The organoleptically tested sterol esters were produced as in Example 1, the mixing ratio of 70% by weight tall oil sterols and 30% by weight rape sterols also being applied to the sterols.

The pure esters mentioned below crystallized very quickly in the mouth and, hence, had an unpleasant mouth feel. Mixtures were more favorable in this regard because they did not show rapid crystallization behavior. Branched fatty acids and fatty acids with a functional group, such as lactic acid, did not show the rapid crystallization behavior either. Accordingly, on the basis of their organoleptic properties, the sterol/lactic acid esters are also suitable for use in foods.

| | |
|---|---|
| Vegapure 100% C2 (acetic acid ester): | 123.8° C. |
| Vegapure 100% C4 (butyric acid ester): | 88.2° C. |
| Vegapure 100% C6 (caproic acid ester): | 68.9° C. |
| Vegapure 100% C8 (caprylic acid ester): | 68.7° C. |
| Vegapure 100% C10 (capric acid ester): | 75.8° C. |
| Vegapure 100% C12 (lauric acid ester): | 81.1° C. |
| Vegapure 100% C8 branched (ethyl hexanoic acid) | 40.5° C. |
| Vegapure 100% lactic acid | 88.7° C. |

Because sterol esters with short fatty acid chain lengths of C2 to C6 have unpleasant sensory properties and since the incorporation of the compounds is promoted by a low melting point, the preferred sterol ester should contain a relatively high percentage of C8 fatty acid.

### Example 4

### Determination of Melting Point

The melting points of the sterol esters with different ratios of C₈ and C₁₀ fatty acids (*similar composition to the sterol esters resulting from Examples 1 and 2) were conventionally determined using melting point capillaries. The sterol esters investigated were produced as in Example 1 using pure octanoic acid and pure decanoic acid, the sterol component containing the mixing ratio of 70% by weight tall oil sterols and 30% by weight rape sterols.

TABLE 1
Melting point determination of sterol C8/C10 fatty acid esters:

| Percentage of C10 fatty acid | |
|---|---|
| chain length (in % by weight, based | |
| on the total weight of the C8 | Melting point |
| and C10 fatty acid used) | in ° C. |
| 0 | 68.7 |
| (corresponds to approx. | |
| 100% by weight sterol | |
| octanoic acid ester) | |
| 10 | 63.0 |
| (ca. 10% sterol ester | |
| C10 and 90% sterol | |
| ester C8% by weight) | |
| 20 | 61.3 |
| 30* | 58.2 |
| 40* | 55.8 |
| 50 | 57.6 |
| 60 | 59.3 |
| 70 | 66.5 |
| 80 | 69.9 |
| 90 | 72.9 |
| 100 | 75.8 |

### Application Examples

### Example 1

### Milk

Incorporation of Sterol-C₈C₁₋₁₀-Fatty Acid Esters 60/40 (from Example 2) in Milk

| | 1) Comparison test | 2) C1 |
|---|---|---|
| Milk, 1.5% fat | 2000 g | 1986.6 g |
| Sterol-C₈C₁₀-fatty acid | | 13.4 g |
| ester 60/40 (Example 2) | | |
| Evaluation after 1 day | Typical in taste | No unpleasant taste |

The milk (10° C.) was poured in and heated to 75° C. Using an Ultra Turrax, the sterol-C₈C₁₋₁₀-fatty acid ester 60/40 (70° C.) was then dispersed in the milk for 30 secs. at 10,000 r.p.m. After homogenization at 150 bar, the preparation was pasteurized at 90° C. and then cooled to 8° C.

### Example 2

### Milk-Based Mixed Beverages

Incorporation of Sterol-C₈C₁₋₁₀-Fatty Acid Esters 60/40 (from Example 2) in a Milk-Based Mixed Beverage

| | 1) Comparison test | 2) C2 |
|---|---|---|
| Milk-based mixed beverage | 1500 g | 1489.95 g |
| Sterol-C₈C₁₀-fatty acid | | 10.05 g |
| ester 60/40 (Example 2) | | |
| Evaluation after 1 day | Typical in taste | No unpleasant taste |

The milk-based mixed beverage (10° C.) was poured in and heated to 75° C. Using an Ultra Turrax, the sterol-C₈C₁₋₁₀-fatty acid ester 60/40 (70° C.) was then dispersed in the milk for 30 secs. at 10,000 r.p.m. After homogenization at 150 bar, the milk-based mixed beverage was pasteurized at 90° C. and then cooled to 8° C. A banana-flavored milk-based mixed beverage (Alois Müller) was used for the tests.

### Example 3

### Yoghurt Preparations

Incorporation of Sterol-C₈C₁₀-Fatty Acid Esters 60/40 (from Example 2) in Solid Yogurt, Drinking Yogurt and Stirred Yogurt

| | 1) Comparison test | 2) C3 |
|---|---|---|
| Milk, 1.5% fat | 3000 g | 2960.1 g |
| Sterol-C₈C₁₀-fatty acid | | 39.9 g |
| ester 60/40 (Example 2) | | |

The milk (10° C.) was poured in and heated to 75° C. Using an Ultra Turrax, the sterol-C₈C₁₀-fatty acid ester 60/40 heated to 70° C. was then dispersed in the milk for 30 secs. at 10,000 r.p.m. After homogenization at 150 bar, the mixture was pasteurized at 90° C. and then cooled to 45° C.

The milk preparation was then inoculated. To this end, a “preliminary” solution of 50 g yogurt cultures (YC 180, Chr. Hansen) and 450 g milk was prepared by stirring. This solution was used in a quantity of 2 ml/liter process liquid. The inoculated preparation was then placed in a conditioning cabinet at 45° C. for fermentation. After reaching a pH of 4.5 to 4.6, the yogurt was cooled (solid yogurt) or had 7% sugar added and was stirred (stirred yogurt) or was re-homogenized at 80-100 bar (drinking yogurt).

### Sensory Impression Results After 1 Day:

Solid yogurt fermented in a container, stirred yogurt and drinking yogurt had no unpleasant taste after incorporation of sterol-C₈C₁₀ fatty acid ester 60/40.

### Example 4

### Vegetable Oils

Incorporation of 5% Sterol-C₈C₁₀-Fatty Acid Ester 60/40 (from Example 2) in Rapeseed Oil

5% sterol-C₈C₁₀-fatty acid ester 60/40 (from Example 2) were dissolved while stirring in rapeseed oil at 70° C. The oil was then cooled to 20° C. The oil mixture was observed for several days to determine whether the sterol-C₈C₁₀-fatty acid ester 60/40 remained in solution. The mixed sample was clear after 1 week, the sterol-C₈C₁₀-fatty acid ester remained in solution.

### Example 5

### Spreading Fats

Incorporation of Sterol-C₈C₁₀-Fatty Acid Ester 60/40 (from Example 2) (75% Total Sterol Content) PF 1106, SR 416/3 in Margarine

| | 1) Comparison | |
|---|---|---|
| | test | 2) C5 |
| Oil phase | | |
| Coconut fat, GS 1 | 30.0% | 30.0% |
| (Cognis) | | |
| Sunflower oil | 49.5% | 39.5% |
| Sterol-C₈C₁₀-fatty | | 10.0% |
| acid ester 60/40 | | |
| (Example 2) | | |
| Monomuls ® 90-35 | 0.3% | 0.3% |
| (Cognis) | | |
| Lecithin | 0.2% | 0.2% |
| β-Carotene (3%) | 0.01% | 0.01% |
| Water phase | | |
| Water | 18.8% | 18.8% |
| Salt | 0.2% | 0.2% |
| Evaluation immediately | Homogeneous, | Homogeneous, slightly glossy |
| after production | slightly | surface, crystallizes out |
| | glossy surface | slightly more quickly |
| Evaluation after 3 days | Typical in taste | Typical in taste, no unpleasant |
| | | taste, no creaming up |

The water phase and the oil phase were separately heated to ca. 60° C.-70° C. The water phase was then slowly added with stirring to the oil phase until an emulsion was formed. The remaining water was then added. The emulsion was then adjusted to pH 5.5 with citric acid and recrystallized with circulation on an ice-cooled metal plate.

### Example 6

### Mayonnaises

Incorporation of Sterol-C₈C₁₀-Fatty Acid Ester 60/40 (from Example 2) in Mayonnaise

| | 1) Comparison | 2) With |
|---|---|---|
| | test | Vegapure MC |
| Lamegin ® ZE 609 (20% paste) | 7.5% | 7.5% |
| (Cognis) | | |
| Water | 26.7% | 26.7% |
| Frigesa ® F 820 (Cognis) | 1.2% | 1.2% |
| Sugar | 5.6% | 5.6% |
| Salt | 1.5% | 1.5% |
| Sunflower oil | 50% | 42% |
| Sterol-C₈C₁₀-fatty acid ester | | 8% |
| 60/40 (Example 2) | | |
| Vinegar | 5% | 5% |
| Mustard | 2.5% | 2.5% |
| Evaluation after 2 days | Typical in taste | Typical, no |
| | | unpleasant taste, |
| | | homogeneous, |
| | | no creaming up |

Water and the emulsifier paste were introduced first. Sugar, salt, stabilizer (Frigesa®) were pre-dispersed with stirring in the oil phase. The oil/solids dispersion was slowly dispersed in the water phase with an Ultra Turrax (level 1). Mustard and vinegar were then added and the whole was re-dispersed with the Ultra Turrax (level 1). For test 2, the sterol-C₈C₁₀-fatty acid ester 60/40 was first dissolved in sunflower oil at 60-70° C. and the sunflower oil was cooled back down.

1-10. (canceled)

11. A composition comprising a combination of at least a first sterol-derived fatty acid esters comprising a sterol-derived moiety and at least one fatty acid moiety with a chain length of 8 carbon atoms, and, at least a second sterol-derived fatty acid ester comprising a sterol-derived moiety and a fatty acid moiety with a chain length of 10 carbon atoms, said composition having a fatty acid distribution of C₈ to C₁₀ of from about 90:10 to about 20:80 (ratio by weight).

12. The composition of claim 11 having a fatty acid distribution of C₈ to C₁₀ of from about 64:36 to about 50:50 (ratio by weight).

13. The composition of claim 11 having a fatty acid distribution of C₈ to C₁₀ of from about 78:22 to about 65:35 (ratio by weight).

14. The composition of claim 11, wherein the fatty acids are selected from the group consisting of octanoic acid, decanoic acid and ethylhexanoic acid.

15. The composition of claim 12, wherein the fatty acids are selected from the group consisting of octanoic acid, decanoic acid and ethylhexanoic acid.

16. The composition of claim 13, wherein the fatty acids are selected from the group consisting of octanoic acid, decanoic acid and ethylhexanoic acid.

17. A food product comprising the composition of claim 11.

18. A food product comprising the composition of claim 12.

19. A food product comprising the composition of claim 13.

20. The food product of claim 17 selected from the group consisting of spreading fats, margarine, butter, vegetable oils, frying fats, peanut butter, mayonnaise, dressings, cereals, bread and confectionery, cakes, wheat bread, rye bread, toast, crispbread, ice cream, desserts, milk products, yogurt, cottage cheese, cream, candies, chocolate, chewing gum, muesli bars, milk beverages, soya beverages, fruit juices, vegetable juices, fermented beverages, noodles, rice, sauces, cheese, spreading cheese, meat and sausages.

21. The food product of claim 18 selected from the group consisting of spreading fats, margarine, butter, vegetable oils, frying fats, peanut butter, mayonnaise, dressings, cereals, bread and confectionery, cakes, wheat bread, rye bread, toast, crispbread, ice cream, desserts, milk products, yogurt, cottage cheese, cream, candies, chocolate, chewing gum, muesli bars, milk beverages, soya beverages, fruit juices, vegetable juices, fermented beverages, noodles, rice, sauces, cheese, spreading cheese, meat and sausages.

22. The food product of claim 19 selected from the group consisting of spreading fats, margarine, butter, vegetable oils, frying fats, peanut butter, mayonnaise, dressings, cereals, bread and confectionery, cakes, wheat bread, rye bread, toast, crispbread, ice cream, desserts, milk products, yogurt, cottage cheese, cream, candies, chocolate, chewing gum, muesli bars, milk beverages, soya beverages, fruit juices, vegetable juices, fermented beverages, noodles, rice, sauces, cheese, spreading cheese, meat and sausages

23. A beverage or milk product comprising 0.1 to 50% by weight of the composition of claim 11.

24. A beverage or milk product comprising 0.1 to 50% by weight of the composition of claim 12.

25. A beverage or milk product comprising 0.1 to 50% by weight of the composition of claim 13.

26. A method for the production of cholesterol-lowering agents comprising incorporating the composition of claim 11.

27. A method for the production of cholesterol-lowering agents comprising incorporating the composition of claim 12.

28. A method for the production of cholesterol-lowering agents comprising incorporating the composition of claim 13.
